(19) United States
(12) Patent Application Publication
(10) Pub. No.: US 2013/0072575 A1
(43) Pub. Date: Mar. 21, 2013

(54) Method and Composition for Treating Pain

(71) Applicants: Jennifer K. Chin, Watchung, NJ (US); Donald Rick, Dumont, NJ (US); Rosemarie Wallace, Old Bridge, NJ (US)

(72) Inventors: Jennifer K. Chin, Watchung, NJ (US); Donald Rick, Dumont, NJ (US); Rosemarie Wallace, Old Bridge, NJ (US)

(73) Assignee: Johnson & Johnson Consumer Companies, Inc., Skillman, NJ (US)

(21) Appl. No.: 13/235,947

(22) Filed: Sep. 19, 2011

(51) Int. Cl. A61K 47/34; A61K 47/12; A61P 29/00; A61K 47/10

(52) U.S. Cl. 514/772.1; 514/772; 514/785

(57) ABSTRACT

Presented are methods of using topical analgesic compositions for rapid relief of muscular aches and pain where the analgesic composition has been stored at a temperature of less than 10° Centigrade prior to use.

### FIELD OF THE INVENTION

The present invention relates to methods of using topical analgesic compositions for rapid relief of muscular aches and pain. More particularly, the invention relates to methods of treating pain by topical application to the skin of a patient of an analgesic composition that has been stored at a temperature of less than 10° Centigrade prior to use.

### BACKGROUND OF THE INVENTION

Local pain can result from any of a variety of causes such as body injury, infection or disease, inflammation, muscle spasm and neuropathy. Examples of conditions that are typically associated with localized pain in the skin or in a tissue or structure near the skin include arthritis, neuropathy, post-herpetic (shingles) conditions, a sore muscle, tendon or ligament, and a local reaction to an insect bite or sting. Typically, a sensation of pain occurs when free nerve endings that constitute pain receptors in the skin or internal tissue are subjected to a mechanical, thermal or chemical stimulus. The stimulus causes the pain receptors to transmit a responsive signal along afferent nerves to the central nervous system and then on to the brain. When pain persists or recurs frequently and treatment provides insufficient relief, in addition to the primary discomfort, the person may be further debilitated by limited function (e.g., of an arthritic joint), reduced mobility, interrupted sleep, and a generally diminished quality of life.

For example, arthritis is medically termed as an inflammation of a joint or joints and is one of a number of diseases and disorders of the skeleton and body system. Arthritis arises from many causes, some well-defined, some still unknown, and it is treated in many different ways. There are two common types, the first of which is inflammatory, of which rheumatoid arthritis is the most commonly acknowledged and a non-inflammatory, second type, most commonly represented by degenerative joint disease, or “wear and tear” arthritis. Degenerative joint disease is a chronic joint disease, often occurring in more elderly people. The joints, whether singly or in multiples, are affected. The primary disease produces symptomatic swelling, pain and stiffness.

Several topical agents (creams, ointments, liniments and the like) have been utilized for the relief of local aches and pain. For mild relief of minor discomfort, there are a variety of non-prescription topical preparations in common use today which contain counter-irritants such as methyl salicylate, camphor, menthol, capsaicin, and eucalyptus. These topical agents are devised for external application to the affected area of the body by applying to the area adjacent to the muscle, joint, ligament or tendon and then rubbing it onto the skin.

The application of ice to the area of pain can also help provide relief in a number of ways. Ice application slows the inflammation and swelling that often accompanies pain, and addressing the inflammation helps reduce the pain. Ice also numbs sore tissues, providing pain relief like a local anesthetic, and slows the nerve impulses in the area, which interrupts the pain-spasm reaction between the nerves. The cold makes the veins in the tissue contract, reducing circulation. Once the cold is removed, the veins overcompensate and dilate and blood rushes into the area. The blood brings with it the necessary nutrients to allow the injured muscles, ligaments and tendons to heal.

Solid sticks and gels are well-established delivery forms for topically applied agents and cosmetics, being particularly useful for lipsticks, deodorants, and antiperspirants. In general, a solid stick consists of an essentially solid matrix that serves as the base for some active ingredient or cosmetic substance. External analgesics, including methyl salicylate, have been formulated in solid stick form using a stearate-alcohol-water matrix.

The search for more effective methods of using topical analgesic compositions for rapid relief of muscular aches and pain than those known at present is a never-ending one. Efforts are constantly being made to find methods which by topical analgesic application will rapidly alleviate pain, such as rheumatic, muscular, and surface aches and pains.

### SUMMARY OF THE INVENTION

The present invention is a method of treating pain by topical application to the skin of a patient of an analgesic composition. The method includes the use of an analgesic composition that has been stored at a temperature of less than 10° Centigrade prior to use. The steps include: 1) store the composition at less than 10° C. for a period of time sufficient to reduce the temperature of the composition, 2) remove the composition from storage, and 3) topically apply the composition to the region of pain.

The invention is also directed to a composition that is capable of being stored at a temperature of less than 10° C. yet maintains properties sufficient for immediate application to the skin to treat pain.

### DETAILED DESCRIPTION OF THE INVENTION

The present invention is a method of treating pain by topical application of an analgesic composition that has been stored at a temperature of less than 10° Centigrade prior to use. The composition is then removed from storage, and topically applied to the region of pain.

The analgesic composition may be stored in an environment with temperatures of less than about 10° C., or less than about 0° C., or less than about −10° C., or less than about −20° C. or in a range from less than about 10° C. to less than about −20° C. It may be stored in a standard refrigerator, which generally has a temperature of less than about 5° C. Alternately, it may be stored in a standard freezer, which generally has a temperature of less than about −15° C. In some embodiments, it may be stored in a portable ice cooler. Prior to use, the analgesic composition is stored in a freezer or refrigerator or reduced temperature environment for a period of time exceeding about 4 hours, or exceeding about 2 hours, or exceeding about 1 hour, or exceeding about 30 minutes, or exceeding about 10 minutes or greater than 10 minutes to over 4 hours. The storage time in the freezer or refrigerator or reduced temperature environment can be up to 20 hours or about 20 hours or up to 48 hours or about 48 hours.

The temperature of the composition should be lowered to about 15 ° C. to about −30 ° C. or from about 10° C. to about −10 ° C. or from about 5° C. to about −5 or about 0° C. When the analgesic composition is ready for use, it is removed from storage, and topically applied to skin in the region of pain. The time period between removal of the analgesic composition and the topical application of it to the region of pain is less than about 10 minutes, or less than about 5 minutes, or less than about 1 minute, or less than about 30 seconds, or less than about 10 seconds, or less than about 5 seconds, or less than about 2 seconds or from less than about 2 seconds to less than about 10 minutes.

The method of application of the analgesic composition to the region of pain depends upon the form of the composition, as well as the type of container holding the composition. Forms of the analgesic composition include, but are not limited to, gel, soft solid, liquid, cream, ointment, aerosol, spray, or stick. The types of containers that hold the composition include, but are not limited to, push-up tube, twist-up tube, flexible tube, pump, non-aerosol spray, wipe, or aerosol can.

For example, if the form of the composition is a cream, and the container holding the composition is a flexible tube, the method of application may be to remove the flexible tube from storage, squeeze the tube to force the analgesic cream composition into the palm of the hand, apply the cream to the region of pain, and massage the cream into the region of pain using the hand. Alternatively, the analgesic cream composition may be directly applied to the region of pain by squeezing the tube to force the cream on to the region of pain, and then massaged into the region of pain by hand, or any of a number of known massage tools.

If the form of the composition is a gel, and the container holding the composition is a twist-up tube, such as those known in the art of deodorant application, the method of application may be to remove the twist-up tube from storage, twist the wheel or other mechanism to force the analgesic gel composition out of the twist-up tube, and directly apply the gel to the region of pain. The gel may then be massaged into the region of pain using the hand, or the end of the twist-up tube.

If the form of the composition is a spray, and the container holding the composition is an aerosol can or spray pump, the method of application may be to remove the aerosol can or spray pump from storage, and directly spraying the analgesic composition onto the skin in the region of pain. The analgesic composition may then be massaged into the region of pain using the hand, or any of a number of known massage tools.

As mentioned, the form of the analgesic composition includes, but is not limited to, gel, soft solid, liquid, cream, ointment, aerosol, spray, or stick. In general, the analgesic composition should have several of the following properties. It should be quick cooling, so that it does not require a long period of storage prior to use. The period of time for the bulk of the analgesic composition to achieve temperatures of less than about 10° C. should not exceed about 4 hours, or about 2 hours, or about 1 hour, or about 30 minutes, or about 15 minutes.

The analgesic composition should also be able to maintain bulk temperatures of less than about 10° C. for the period of time between removal of the analgesic composition from storage and the topical application of the composition to the region of pain. The period of time for the analgesic composition to maintain bulk temperatures of less than about 10° C. should exceed about 60 minutes, or about 30 minutes, or about 10 minute, or about 5 minutes, or about 1 minute, so that the user reaps the benefits of cold composition temperatures during use.

This allows for the application of the analgesic composition to the skin of a user immediately after removal from the cold storage temperatures of less than 10° C. or lower, within 1 minute of the removal from the cold storage temperature, within 5 minutes of removal from the cold storage temperatures or up to 60 minutes after removal from the cold storage temperatures.

One other general property of the analgesic composition is that after use, and when placed back into cold storage, the composition should not show phase separation. A phase separation will limit the ability to reuse the composition. The composition must also be capable of storage in temperatures of less than about 5° C. yet maintain a consistency for topical application and maintain that consistency without phase separation through multiple warming/cooling cycles.

Generally, the components of the analgesic composition should serve a function, or functions, needed to properly store and/or use the composition effectively. If, for example, the analgesic composition is in the form of a gel, the components used include at least one external analgesic, and at least one solubilizer to act as a carrier for the analgesic. The composition may optionally include at least one stabilizer to prevent phase separation of the composition during warming/cooling cycles, and one or more skin conditioners, humectants, fragrances, viscosity adjusters, preservatives, and pH adjusters.

A number of external analgesics may be used in the present invention. The external analgesics should be in an effective therapeutic amount in the composition. The external analgesics, and their effective therapeutic amounts, include, but are not limited to, methyl salicylate (10 to 60 percent), ammonia solution (1.0 to 2.5 percent), turpentine oil (6.0 to 50 percent), camphor (3 to 11 percent), menthol (1.25 to 16 percent), histamine dihydrochloride (0.025 to 0.10 percent), capsaicin (0.025 to 0.25 percent), capsicum containing 0.025 to 0.25 percent capsaicin, capsicum oleoresin containing 0.025 to 0.25 percent capsaicin, allyl isothiocyanate (0.5 to 5.0 percent) and methyl nicotinate (0.25 to 1 percent). Mixtures of these are also useful. These actives are both compatible with the vehicle as well as being capable of delivering perceptible warmth to the skin and underlying tissues, muscles and joints. In some embodiments, menthol is used as the external analgesic.

Solubilizers which may be used in the present invention include, but are not limited to water, alcohols such as ethanol, methanol, propanol, propylene glycol and benzyl alcohol, butylene glycol, cyclopentasiloxane, cyclomethicone, dibutyl adipate, butyloctyl salicylate, dibutyl sebacate, diethylene glycol, dipropylene glycol, hexanediol, isopentane, isopropyl alcohol, methyl lactate, mineral oil, pentylene glycol, propanediol, hexylene glycol, ethoxydiglycol, vegetable oil (Canola oil), castor oil, sweet almond oil, wheat germ oil, and mixtures thereof. The solubilizers are present in amounts of 70 (or about 70) to 92 (or about 92) percent by weight of the total composition for stick compositions or from 85 to 90 percent (or about 85 to about 90 percent) by weight of the total composition for gel compositions or from 80 to 95 percent (or about 80 to about 95percent) by weight of the total composition for liquid compositions.

Propylene glycol, 1,3-propanediol hexylene glycol, dipropylene glycol, tripropylene glycol, glycerin, ethanol, propylene glycol methyl ether, dipropylene glycol methyl ether, dipropylene glycol, tripropylene glycol, ethanol, n-propanol, n-butanol, t-butanol, 2-methoxyethanol, 2-ethoxyethanol, ethylene glycol, isopropanol, isbutanol, 1,4-butylene glycol, 2,3-butylene glycol, 2,4-dihydroxy-2-methylpentane, trimethylene glycol, 1,3-butanediol, 1,4,-butanediol, 1,2-hexanediol and mixtures thereof can be used in freezing point lowering or depressing amounts in the gel composition so that the gel can be applied to the skin at temperatures found in refrigerators and freezers. Preferred ingredients to lower or depress the freezing point are propylene glycol, 1,3-propanediol and mixtures thereof.

The freezing point lowering compound can be added to the compositions in amounts from 20 (or about 20) to 75 (or about 75) percent by weight or from 25 (or about 25) to 50 (or about 50) percent by weight of the compositions.

At least one stabilizer, to prevent phase separation of the composition during warming/cooling cycles, may be incorporated into the analgesic compositions. These would include, without limitation, the nonionic surfactants such as the poly-alkanolamines, e.g., triethanolamine, polyethylene glycol stearate, polyethylene glycol laurate, polyoxyethylene and polyoxypropylene compounds (e.g., as derivatives of sorbitan and fatty alcohol ethers and esters, polyoxypropylene-polyoxyethylene block copolymers), fatty acid esters of polyhydric alcohols and amine oxides; anionic surfactants, such as alkyl carboxylates, acyl lactylates, sulfuric acid esters (e.g., sodium lauryl sulfate), ester-linked sulfonates, and phosphated ethoxylated alcohols. In some embodiments, nonionic surfactants like ethoxylated sorbitan esters (for example TWEEN 60-LQ-(AP), available from Croda Inc, Edison, N.J.), may be used as a stabilizer. In other embodiments, polyoxyethlyene, polyoxypropylene block polymers, such as poloxamers available from under the trade name PLURONIC from BASF, Florham Park, N.J.) may be used. Stabilizers may be employed in amounts of about 0.1 to about 5 percent by weight of the total composition.

Skin conditioners, which are typically categorized as emollients, may also be included in the composition. These ingredients serve to aid in deposition of the composition onto the skin, as well as to remove any undesired residue from the skin after use. Suitable emollients would include octyl isononanoate, fatty acid esters such as cetyl palmitate, diisopropyl adipate, isopropyl isostearate, isostearyl isostearate, lauryl lactate, polyalkylene glycols, isopropyl myristate (the ester of isopropanol and myristic acid), and mixtures thereof. In some embodiments, octyl isononanoate (for example DERMOL 89, available from Alzo International Incorporated Sayreville, N.J.), may be used as a skin conditioner Skin conditioners may be employed in amounts of about 0.25 to about 2 percent by weight of the total composition.

Suitable humectants include glycerin, propylene glycol, polyethylene glycol and mixtures thereof. Preferably, glycerin or sorbitol is used. They are generally present in amounts of about 0.5 to about 6 percent by weight of the total composition.

Fragrances include camphor, menthol, and eucalyptus.

Viscosity adjusters include Carbolpol Ultrez 10 (Acrylic acid copolymer) from The Lubrizol Corporation, Wickliffe, Ohio, and urethane based emulsifying agents such as DERMOTHIX-75 (Alzo International Incorporated, Sayreville, N.J.). DERMOTHIX-75 liquid is a nonionic surfactant composed of 50% actives, a nonionic and water. The INCI name is Disteareth-75 IPDI (and) PEG-7 caprylate/caprate (and) aqua, where IPDI stands for isophorone diisocyanate.

There are many reasons to use a pH adjuster, but they are generally employed whenever a product is too acidic or too basic. Materials that may be used as pH adjusters include, but are not limited to lactic acid, citric acid, triethanolamine, sodium hydroxide, and ammonium hydroxide. In some embodiments, AMP (Amino-ethyl Propanol), available from Angus Chemical Company, Buffalo Grove, Ill., may be used.

A variety of bacteristats or preservatives may be incorporated providing they are compatible with the acid-soap gelling agent. Substituted phenols and derivatives thereof are one such class of preservatives which may be added. Examples include the chloro-substituted phenoxy phenols, such as 5-chloro-2-(2,4-dichlorophenoxy)phenol; 3,4,4-trichlorocarbanilide and bithionol, hexachlorophene, triclosan, dichlorophene, among others; mercury derivatives, such as phenylmercuric acetate; quaternaries, such as benzethonium chloride, benzalkonium chlorides and cetyl trimethyl ammonium bromide; acids, such as sorbic acid, and a variety of other preservatives.

To form the analgesic gel composition, the components of the composition can be sequentially added to a mixer until the gel is formed. In some cases, the components can be split into two premixes, and the premixes combined to form the gel. Another form of the analgesic composition is a stick. Cosmetic stick compositions are well known in the art. Antiperspirants, deodorants, lipsticks and the like all use stick, or gel stick technology. The stick compositions include a delivery system comprising about 75 to about 95 percent by weight solubilizer; about 4 to about 10 percent by weight of an alkali metal salt of a saturated fatty acid gelling agent, and at least one external analgesic. The composition may optionally include at least one stabilizer, and one or more skin conditioners, humectants, fragrances, viscosity adjusters, preservatives, and pH adjusters.

Anhydrous/wax stick compositions include a delivery system comprising about 60 to about 75 percent by weight solubilizer; and 10 to 25 percent structurant.

Solubilizers, external analgesics, stabilizers, skin conditioners, humectants, fragrances, viscosity adjusters, preservatives, and pH adjusters have all been discussed above.

The useful gelling agents include the alkali metal stearates and palmitates. For example, sodium stearate, potassium stearate, sodium palmitate, potassium palmitate, sodium potassium stearate, beeswax, microcrystalline wax, candelilla wax, carbowax, carnauba wax among others, are particularly beneficial. The term “sodium stearate” is herein used to connote the sodium salt of a mixture of fatty acids, of which stearic acid and palmitic acid predominate and with relatively small proportions of closely related fatty acids. In some embodiments, the stick composition may be made where the gelling agents are formed in situ during the formulating of the composition. To do so, aqueous alkali such as sodium or potassium hydroxide are added to a warm aqueous/alcohol solution of the fatty acid, e.g., stearic or palmitic acid. For example, aqueous sodium hydroxide may be added to a solution of solubilizer and topical analgesic, and mixed at about 50 to about 75° C. The solution is then allowed to cool to about 45 to about 50° C., at which time the analgesic composition is poured into the desired mold and further cooled until it solidifies.

The types of containers that hold the composition include, but are not limited to, push-up tube, twist-up tube, flexible tube, pump, or aerosol can.

Push-up tubes are containers in the form of tubes with uniform cross-sections (round, ovoid, square, rectangular), formed of polymers (like polystyrene) with ribbed screw caps and plastic bases. To dispense contents of these containers, simply push on the plastic bases of the containers, which often feature molded text reading “Push Up”. Push-up tubes are often used to package deodorants, as well as sunscreens, solid perfumes and other bath and body care products.

Twist-up tubes are containers in the form of tubes with uniform cross-sections (round, ovoid, square, rectangular), formed of polymers (like polystyrene) with ribbed screw caps and equipped with a suitable twist-up device inserted into the bottom thereof. The present invention will be better understood from a consideration of the following illustrative examples, in which all percentages of ingredients are expressed in percent by weight of the total composition unless otherwise indicated.

### EXAMPLES

### Example 1

### Stick Formulations

Topical analgesic stick formulations were made. The formulas are shown on Table 1.

TABLE 1
Stick Formulas (Components are in grams).

| | Stick Formula | | | | | | |
|---|---|---|---|---|---|---|---|
| | 1 | 2 | 3 | 4 | 5 | 6 | 7 |
| Water | 299.5 | 110 | 132 | 143 | 143 | 143 | 32.5 |
| Ethanol | — | — | — | — | — | — | 195 |
| Propylene | 50 | 316.5 | 408.6 | 422.5 | 395.2 | 372.58 | 302.38 |
| Glycol | | | | | | | |
| Propanediol | 50 | — | — | — | — | — | — |
| Butylene | 25 | 25 | — | — | — | — | — |
| Glycol | | | | | | | |
| Stearic Acid | — | 24.5 | 29.4 | 31.85 | 31.85 | 52 | 52 |
| Sodium | 35 | — | — | — | — | — | — |
| Stearate | | | | | | | |
| Menthol | 35 | 15 | 18 | 39 | 65 | 65 | 39 |
| Isopropyl | 5 | 5 | 6 | 6.5 | 6.5 | 6.5 | 19.5 |
| myristate | | | | | | | |
| (IPM) | | | | | | | |
| Poloxamer | — | — | 1.2 | 1.3 | 2.6 | 2.6 | 1.3 |
| 407 | | | | | | | |
| Camphor | — | — | — | 0.65 | 0.65 | 0.65 | 0.65 |
| Sodium | — | 4 | 4.8 | 5.2 | 5.2 | 7.67 | 7.67 |
| Hydroxide | | | | | | | |
| Tetrasodium | 0.5 | — | — | — | — | — | — |
| EDTA | | | | | | | |
| TOTAL | 500 | 500 | 600 | 650 | 650 | 650 | 650 |

Topical analgesic sticks were made from formula 1 as follows:
1. Added all ingredients into a 1000 milliliter metal beaker.
2. Placed metal beaker in water bath heated to 80° C.
3. Mixed with overhead mixer until mixture is clear.
4. The mixture was cooled, and poured into 3 ounce (88.7 ml) containers when temperature reached between 45° C. and 50° C.

Topical analgesic sticks were made from formulas 2, 4, 5, and 6 as follows:
1. Heated water bath to 70° C.
2. Added all propylene glycol and IPM into a 1000 milliliter metal beaker, and placed metal beaker into the water bath.
3. Added 30 grams water to the 1000 milliliter metal beaker and placed the rest into a 150 milliliter glass beaker. Placed metal beaker into the water bath.
4. Added sodium hydroxide into the 50 milliliter glass beaker of water, and mixed the sodium hydroxide until it is dissolved.
5. Lowered the water bath temperature to 65° C., and added all the stearic acid to the 1000 milliliter metal beaker. Mixed until all stearic acid dissolved.
6. Once mixture in beaker cleared, lowered the water bath temperature to 58° C.
7. Added all the menthol and camphor to the 1000 milliliter metal beaker, and mixed until all dissolved.
8. Added the sodium hydroxide solution from the 50 milliliter glass beaker into the 1000 milliliter metal beaker and mix for 10 minutes.
9. At this point, the mixture in the 1000 milliliter metal beaker was clear.
10. The mixture was cooled, and poured into 3 ounce (88.7 ml) containers when temperature reached between 45° C. and 50° C.

Topical analgesic sticks were also made from formulas 3 and 7. The same steps were used for formula 3, except in step 7 no camphor was added to the 1000 milliliter metal beaker. For formula 7, except in step 3 except both water and ethanol were added to the 1000 milliliter metal beaker.

A wax-based topical analgesic stick formulation was also made. The formula is shown on Table 2.

TABLE 2
Wax-Based Stick Formula (Components are in grams).

| Ingredient | Formula 8 |
|---|---|
| Menthol | 35 |
| Vegetable Oil (Canola Oil) | 155 |
| Beeswax | 75 |
| Cocoa Butter | 40 |
| Castor Oil | 80 |
| Sweet Almond Oil | 55 |
| Wheat Germ Oil | 40 |
| Tocopherol, Vitamin E | 12.5 |
| Fragrance | 0 |
| Total | 492.5 |

Formulas 3, 4, 5, 6, and 7 formed good quality sticks which easily pushed out of the 3-ounce containers. Formula 1 stuck to the sides of package and could not be pushed up, while in Formula 2, the menthol crystallized out and dropped to the bottom half of the containers after freezing. The wax-based sticks (formula 8) did not display good slip, though it is contemplated that slip can be modified with different emollients or skin conditioners.

Formula 8 was tested for how long it took to cool down in a cold environment, as well as how long it took the gels to warm up once removed from a cold environment. In the first test, 88.7 ml of wax-based stick formula 8 was placed on a lab bench at room temperature. The gel was moved into a freezer at −30° C. The temperature of the gel was measured over the next 2 hours. Table 3 shows the temperature versus time for the specimen.

TABLE 3
Time versus temperature for cooling wax-based
stick formula 8 in a freezer.

| Time (min) | Temperature (° C.) |
|---|---|
| 0 | 22 |
| 15 | 10 |
| 30 | 0 |
| 45 | −7.5 |
| 60 | −12.5 |
| 75 | −15 |
| 90 | −17 |
| 120 | −20 |

The table shows the formula cooled from room temperature to below the freezing point of water (0° C.) in about 30 minutes.

Formula 8 was tested for how long it took to warm up to room temperature once it was once removed from a cold environment. In this test, 88.7 ml of wax-based stick formula 8 was removed from the freezer mentioned above, and placed on a lab bench at room temperature. The temperature of the gel was measured over the next 2 hours. Table 4 shows the temperature versus time for the specimen.

TABLE 4
Time for wax-based stick formula 8 to thaw after being removed
from the freezer to room temperature.

| TIME (min) | Temperature (° C.) |
|---|---|
| 0 | −30 |
| 10 | −17.5 |
| 20 | −10 |
| 30 | −2.5 |
| 40 | 2.5 |
| 50 | 7.5 |
| 60 | 10 |
| 90 | 15 |
| 120 | 17.5 |

The table shows the formula warmed from freezer temperature (−30° C.) to 17.5° C. in about 2 hours.

TABLE 5
Time for formulas 3 and 4 to cool in a freezer and to thaw after
being removed from the freezer to room temperature

| | Formula 3 | Formula 3 | | |
|---|---|---|---|---|
| | Cooling | Thawing | Formula 4 | Formula 4 |
| Time (min) | (° C.) | (° C.) | Cooling (° C.) | Thawing (° C.) |
| 0 | 21.3 | −23.1 | 21.0 | −22.7 |
| 15 | 16.6 | −9.8 | 13.2 | −12.4 |
| 30 | 7.9 | 0.4 | 4.5 | −2.6 |
| 45 | 0.9 | 6.9 | −2.1 | 4.1 |
| 60 | −4.8 | 11.1 | −7.3 | 8.6 |
| 75 | −9.5 | 13.9 | −11.2 | 11.5 |
| 105 | −15.7 | 19.5 | −15.8 | 14.4 |
| 120 | −18.0 | 18.7 | −17.1 | 16.4 |

### Example 2

### Gel Formulations

Topical analgesic gel formulations were made. The formulas are shown on Table 6.

TABLE 6
Gel Formulas (Components are in grams).

| | Gel Formula | | | | |
|---|---|---|---|---|---|
| | 9 | 10 | 11 | 12 | 13 |
| SDA 3C 190 Proof Ethanol | 100 | 175.0 | 150.0 | 150.0 | 150.0 |
| Racemic Menthol, | 20 | 25.0 | 25.0 | 25.0 | 25.0 |
| USP/FCC | | | | | |
| Camphor Powder Synthetic, | 0.4 | 0.50 | 0.50 | 0.50 | 0.50 |
| USP | | | | | |
| Tween 60-LQ-(AP) | — | 5.0 | 5.0 | 5.0 | 5.0 |
| Dermothix 75 Liquid | — | 2.5 | 2.5 | 2.5 | — |
| Dermol 89 | — | 2.5 | 2.5 | 2.5 | 5.0 |
| Purified Water | 262.4 | 248.0 | 273.0 | 248.0 | 273.0 |
| Glycerin 99% USP | 12 | 10.0 | 10.0 | 10.0 | 10.0 |
| Propylene Glycol | — | 25.0 | 25.0 | 50.0 | — |
| 1,3-Propanediol | — | — | — | — | 25.0 |
| Carbopol Ultrez 10 | 3.6 | 4.5 | 4.5 | 4.5 | 4.5 |
| AMP 95 | 1.6 | 2.0 | 2.0 | 2.0 | 2.0 |
| TOTAL | 400 | 500.0 | 500.0 | 500.0 | 500.0 |

Topical analgesic gel sticks were made from formula 9 as follows:
1. Added all propylene glycol and glycerin into a 1000 milliliter glass beaker.
2. Added 20 grams of water into a 50 milliliter glass beaker.
3. Added remaining water to the 1000 milliliter glass beaker and mix in an overhead mixer at 200 rpm.
4. Added AMP into the 50 milliliter glass beaker of water, and mixed the AMP until it is dissolved.
5. Added all the Carbopol to the 1000 milliliter glass beaker. Mixed until all Carbopol dissolved.
6. Increased the mixing speed to 230 rpm and mix for 30 minutes.
7. Added ethanol, menthol, camphor, Tween, Dermothix, and Dermal into a separate 400 milliliter glass beaker.
8. Added a magnetic bar to the 400 milliliter glass beaker and mix over a stirrer.
9. When the menthol and camphor were dissolved, added the mixture to the 1000 milliliter glass beaker.
10. Mixed for 15 minutes and added AMP solution from the 50 milliliter glass beaker.
11. Changed mixing blade to a paddle and mixed for 5 minutes or until the gel was uniform.
12. The mixture was poured into 3 ounce (88.7 ml) containers.

Topical analgesic gels were made from formulas 10, 11, and 12 as follows:
1. Added all propylene glycol and glycerin into a 1000 milliliter glass beaker.
2. Added 20 grams of water into a 50 milliliter glass beaker.
3. Added remaining water to the 1000 milliliter glass beaker and mix in an overhead mixer at 200 rpm.
4. Added AMP into the 50 milliliter glass beaker of water, and mixed the AMP until it is dissolved.
5. Added all the Carbopol to the 1000 milliliter glass beaker. Mixed until all Carbopol dissolved.
6. Increased the mixing speed to 230 rpm and mix for 30 minutes.
7. Added ethanol, menthol, camphor, Tween, Dermothix, and Dermal into a separate 400 milliliter glass beaker.
8. Added a magnetic bar to the 400 milliliter glass beaker and mix over a stirrer.
9. When the menthol and camphor were dissolved, added the mixture to the 1000 milliliter glass beaker.
10. Mixed for 15 minutes and added AMP solution from the 50 milliliter glass beaker.
11. Changed mixing blade to a paddle and mixed for 5 minutes or until the gel was uniform.
12. The mixture was poured into 3 ounce (88.7 ml) containers.

Topical analgesic gel was also made from formula 13. The same steps were used, except in step 1, 1,3-Propanediol was added to the 1000 milliliter glass beaker instead of propylene glycol.

Formulas 10, 11, 12, and 13 formed good quality gels. Formula 9, when in the freezer contained ice crystals in the gel, and had a sponge-like consistency.

Formula 11 was tested for how long it took to cool down in a cold environment, as well as how long it took the gels to warm up once removed from a cold environment. In the first test, 88.7 ml of gel formula 11 was placed on a lab bench at room temperature. The gel was moved into a freezer at −30° C. The temperature of the gel was measured over the next 2 hours. Table 7 shows the temperature versus time for the specimen.

TABLE 7
Time versus temperature for cooling Gel formula 11 in a freezer.

| Time (min) | Temperature (° C.) |
|---|---|
| 0 | 22 |
| 15 | 12.5 |
| 30 | 3 |
| 45 | −3 |
| 60 | −7.5 |
| 75 | −10 |
| 105 | −17 |
| 120 | −20 |

The table shows the formula cooled from room temperature to below the freezing point of water (0° C.) in about 40 minutes.

Formula 11 was tested for how long it took to warm up to room temperature once it was once removed from a cold environment. In this test, 88.7 ml of gel formula 11 was removed from the freezer mentioned above, and placed on a lab bench at room temperature. The temperature of the gel was measured over the next 2 hours. Table 8 shows the temperature versus time for the specimen.

TABLE 8
Time for gel formula 11 to thaw after being removed from
the freezer to room temperature.

| TIME (min) | Temperature (° C.) |
|---|---|
| 0 | −30 |
| 10 | −20 |
| 20 | −11 |
| 30 | −5 |
| 40 | 0 |
| 50 | 3 |
| 60 | 5 |
| 90 | 15 |
| 120 | 17.5 |

The table shows the formula warmed from freezer temperature (−30° C.) to 17.5° C. in about 2 hours.

### Example 3

### Spray Formulations

Spray formulations were made. The formulas are shown on Table 9.

TABLE 9
Spray Formulas (Components percent weight)

| | Spray Formula | | | | |
|---|---|---|---|---|---|
| | 14 | 15 | 16 | 17 | 18 |
| SDA 3C 190 Proof | 35.00 | 35.00 | 30.00 | 30.00 | 56.00 |
| Ethanol | | | | | |
| Racemic Menthol, | 5.00 | 5.00 | 5.00 | 5.00 | 10.00 |
| USP/FCC | | | | | |
| Purified Water | 53.00 | 52.78 | 57.00 | 57.78 | 0.00 |
| Glycerin 99% USP | 2.00 | 2.00 | 2.00 | 2.00 | 4.00 |
| Propylene Glycol | 5.00 | 5.00 | 5.00 | 5.00 | 25.00 |
| Polysorbate 20 | | | 1.00 | | |
| Poloxamer 407 | | 0.23 | | 0.23 | |
| Butylene Glycol | | | | | 5.00 |
| | 100.00 | 100.00 | 100.00 | 100.00 | 100.00 |

Process for Making Spray Formulas:
1. Add ethanol and menthol to a 200 milliliter glass beaker
2. Add a magnetic bar to the glass beaker and mix over a stirrer until menthol is dissolved
3. Add water, glycols and glycerin into a 400 milliliter glass beaker.
4. Mixed with overhead for 5 minutes.
5. Add surfactant and mix with overhead for 10 minutes
6. When both mixtures ethanol/menthol and glycol/glycerin are uniform, add ethanol/menthol mixture to glycol/glycerin.
7. Mix with overhead mixture for 5 minutes or until uniform
8. The mixture was poured into 2 ounce containers.

After storage at 1 week at 25 ° C. and 1 week at −20 ° C., only Formula 18 remained a clear liquid capable of application to the skin as a spray. The remaining formulations separated and partially froze at −20 ° C.

It is apparent that the inventive methods are unique in their ability to deliver effective amounts of topical analgesic compositions for rapid relief of muscular aches and pain. This invention may be embodied in other forms or carried out in other ways without departing from the spirit or essential characteristics thereof. The present disclosure is therefore to be considered as in all respects illustrative and not restrictive, the scope of the invention being indicated by the appended claims, and all changes which come within the meaning and range of equivalency are intended to be embraced therein.

What is claimed is:

1. A method of treating pain by topical application of an analgesic composition to the skin of a patient comprising
a) placing an analgesic composition in an environment with a temperature of less than 10° Centigrade,
b) keeping said analgesic composition in said environment for a period of time sufficient to reduce the temperature of said composition
c) removing said composition from said environment, and
d) applying said composition topically to the region of pain.

2. The method according to claim 1 wherein said analgesic composition is placed in an environment with a temperate of less than 0° Centigrade.

3. The method according to claim 2 wherein said analgesic composition is placed in an environment with a temperate of less than −10° Centigrade.

4. The method according to claim 1 wherein the temperature of said composition during the application topically to the region of pain is from about 15° Centigrade to about −30° Centigrade.

5. The method according to claim 1 wherein said analgesic composition is in a form selected from the group consisting of gel, soft solid, liquid, cream, ointment, or aerosol.

6. The method according to claim 1 wherein said composition is stored in a container selected from the group consisting of a push-up tube, a twist-up tube, flexible tube, pump, non-aerosol spray, wipe, or aerosol can.

7. The method according to claim 1 wherein the step of applying said composition comprises rubbing said composition onto the skin.

8. The method according to claim 1 wherein the step of applying said composition comprises spraying said composition onto the skin.

9. The method according to claim 1 wherein said analgesic composition comprises a solubilizer selected from the group consisting of propylene glycol, butylene glycol, and mixtures thereof.

10. The method according to claim 9 wherein said analgesic composition further comprises a stabilizer selected from the group consisting of poloxamer.

11. The method according to claim 1 wherein said analgesic composition further comprises a gelling agent selected from the group consisting of alkali metal stearates and alkali metal palmitates.

12. The method according to claim 11 wherein said alkali metal stearates and alkali metal palmitates are formed by mixing a fatty acid with an alkali composition.

13. The method according to claim 1 wherein said period of time comprises about 10 minutes to about 48 hours.

14. The method according to claim 13 wherein said period of time comprises about 1 hour to about 20 hours.

15. The method according to claim 14 wherein said period of time comprises about 2 hours to about 4 hours.

16. A method of treating pain by topical application of an analgesic composition to the skin of a patient comprising
a) placing an analgesic composition in an environment with a temperature of less than 10° Centigrade until said composition reaches a temperature from about 15° Centigrade to about −30° Centigrade,
b) removing said composition from said environment, and
c) applying said composition topically to the region of pain.

17. The method according to claim 16 wherein said analgesic composition reaches a temperature of about 10° Centigrade to about −10° Centigrade.

18. The method according to claim 17 wherein said analgesic composition reaches a temperature of about 5° Centigrade to about −5° Centigrade.

19. The method according to claim 16 wherein said analgesic composition is in a form selected from the group consisting of gel, soft solid, liquid, cream, ointment, or aerosol.

20. The method according to claim 16 wherein said composition is stored in a container selected from the group consisting of a push-up tube, a twist-up tube, flexible tube, pump, non-aerosol spray, wipe, or aerosol can.

21. The method according to claim 16 wherein the step of applying said composition comprises rubbing said composition onto the skin.

22. The method according to claim 16 wherein the step of applying said composition comprises spraying said composition onto the skin.

23. The method according to claim 16 wherein said analgesic composition comprises a solubilizer selected from the group consisting of propylene glycol, butylene glycol, and mixtures thereof

24. The method according to claim 23 wherein said analgesic composition further comprises a stabilizer selected from the group consisting of poloxamer.

25. The method according to claim 16 wherein said analgesic composition further comprises a gelling agent selected from the group consisting of alkali metal stearates and alkali metal palmitates.

26. The method according to claim 25 wherein said alkali metal stearates and alkali metal palmitates are formed by mixing a fatty acid with an alkali composition.

27. The method according to claim 16 wherein said analgesic composition is placed in an environment with a temperature of less than 0° Centigrade.

28. The method according to claim 27 wherein said analgesic composition is placed in an environment with a temperature of less than −10° Centigrade.
